(19) United States
(12) Patent Application Publication
(10) Pub. No.: US 2024/0050004 A1
(43) Pub. Date: Feb. 15, 2024

(54) STATE MANAGEMENT DEVICE, STATE MANAGEMENT METHOD, AND RECORDING MEDIUM

(71) Applicant: NEC Corporation, Minato-ku, Tokyo (JP)

(72) Inventor: Toshiyuki TAMURA, Tokyo (JP)

(73) Assignee: NEC Corporation, Minato-ku, Tokyo (JP)

(21) Appl. No.: 17/766,343

(22) PCT Filed: Oct. 5, 2020
(86) PCT No.: PCT/JP2020/037705

§ 371 Date: Apr. 4, 2022

(30) Foreign Application Priority Data
Oct. 8, 2019 (JP) 2019-185118

(51) Int. Cl. A61B 5/16; A61B 5/00

(52) U.S. Cl. CPC A61B 5/165; A61B 5/6803

(57) ABSTRACT

A state management device acquires biometric information pertaining to a living body of a measurement subject from a wearable terminal worn by the measurement subject. The state management device specifies stability promoting information for promoting stability of a state of the measurement subject, based on the biometric information and a characteristic of the measurement subject. The state management device outputs the stability promoting information.

### TECHNICAL FIELD

The present invention relates to a state management device, a state management method, and a recording medium.

### BACKGROUND ART

There is a demand to appropriately manage the state of a living body of a measurement subject such as the health state. For example, Patent Document 1 discloses a technique for managing the state of a living body of a measurement subject such as a health state.

### PRIOR ART DOCUMENTS

### Non-Patent Documents

Patent Document 1: PCT International Publication No. WO 2018/078222

### SUMMARY OF THE INVENTION

### Problem to be Solved by the Invention

In the above-mentioned technique, it is required to perform stability promotion of a measurement subject to a higher degree.

An example object of the present invention is to provide a state management device, a state management method, and a recording medium that solve the aforementioned problem.

### Means for Solving the Problem

According to a first example aspect of the present invention, a state management device includes: an acquisition means for acquiring biometric information pertaining to a living body of a measurement subject from a wearable terminal worn by the measurement subject; a stability promoting information specifying means for specifying stability promoting information for promoting stability of a state of the measurement subject, based on the biometric information and a characteristic of the measurement subject; and an output means for outputting the stability promoting information.

According to a second example aspect of the present invention, a state management device includes: an acquisition means for acquiring state information pertaining to a state of a measurement subject from a wearable terminal worn by the measurement subject; a stability promoting information specifying means for specifying a coping method for promoting stability of the state of the measurement subject, based on the state information and a characteristic of the measurement subject; and an output means for outputting the coping method.

According to a third example aspect of the present invention, a state management method includes: acquiring biometric information pertaining to living body of a measurement subject from a wearable terminal worn by the measurement subject; specifying stability promoting information for promoting stability of a state of the measurement subject, based on the biometric information and a characteristic of the measurement subject; and outputting the stability promoting information.

According to a fourth example aspect of the present invention, a state management method includes: acquiring state information pertaining to a state of a measurement subject from a wearable terminal worn by the measurement subject; specifying a coping method for promoting stability of the state of the measurement subject, based on the state information and a characteristic of the measurement subject; and outputting the coping method.

According to a fifth example aspect of the present invention, a recording medium stores a program for causing a computer of a state management device to execute: acquiring biometric information pertaining to a living body of a measurement subject from a wearable terminal worn by the measurement subject; specifying stability promoting information for promoting stability of a state of the measurement subject, based on the biometric information and a characteristic of the measurement subject; and outputting the stability promoting information.

According to a sixth example aspect of the present invention, a recording medium stores a program for causing a computer of a state management device to execute: acquiring state information pertaining to a state of a measurement subject from a wearable terminal worn by the measurement subject; specifying a coping method for promoting stability of the state of the measurement subject, based on the state information and a characteristic of the measurement subject; and outputting the coping method.

### Effect of Invention

According to an example embodiment of the present invention, it is possible to perform stability promotion of a measurement subject to a higher degree.

### BRIEF DESCRIPTION OF THE DRAWINGS

FIG. 1 is a diagram showing an outline of the state management system according to an example embodiment of the present invention.

FIG. 2 is a diagram showing a hardware configuration of a wearable terminal according to the example embodiment of the present invention.

FIG. 3 is a diagram showing a hardware configuration of the state management device, the external device, and the mobile terminal according to the example embodiment of the present invention.

FIG. 4 is a function block diagram of the state management device according to the example embodiment of the present invention.

FIG. 5 is a diagram showing the processing flow of the state management system according to the first example embodiment.

FIG. 6 is a diagram showing the processing flow of the state management system according to the second example embodiment.

FIG. 7 is a diagram showing the processing flow of the state management system according to the fourth example embodiment.

FIG. 8 is a diagram showing a state management device according to another example embodiment of the present invention.

FIG. 9 is a diagram showing a first processing flow of the state management device shown in FIG. 8.

FIG. 10 is a diagram showing a second processing flow of the state management device shown in FIG. 8.

### EXAMPLE EMBODIMENT

Hereinbelow, the state management system according to an example embodiment of the present invention will be described with reference to the drawings.

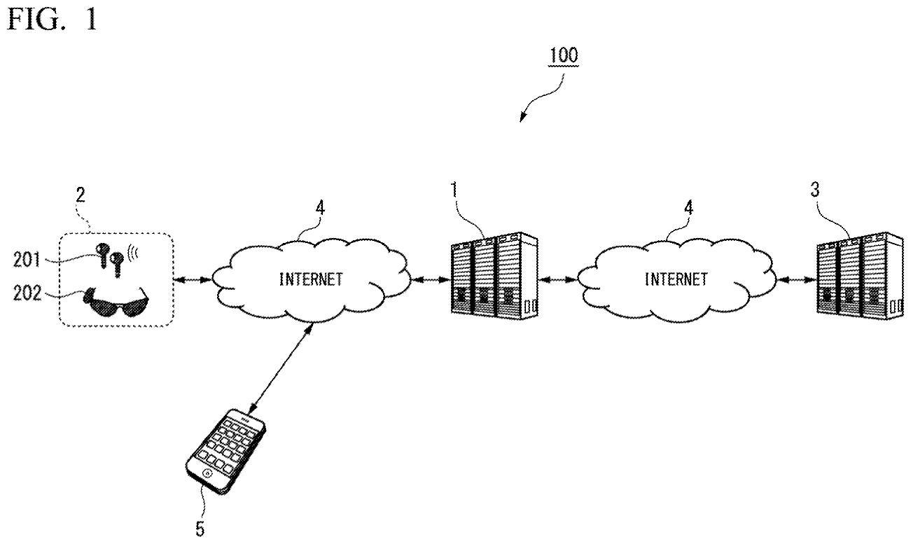

FIG. 1 is a diagram showing an outline of the state management system.

As shown in FIG. 1, a state management system 100 is configured by connecting at least a state management device 1 and a wearable terminal 2 via a communication network such as the Internet 4. An external device 3 and a mobile terminal 5 are further connected to the state management system 100 shown in FIG. 1. The external device 3 is connected to the state management device 1 via a communication network such as the Internet 4. The mobile terminal 5 is also connected to the state management device 1 via a communication network such as the Internet 4. The wearable terminal 2 is worn by a patient in this example embodiment. The mobile terminal 5 is carried by a user such as a doctor, a nurse, or a caregiver who provides support to the patient. As an example, the external device 3 may be a device installed for a manager different from the manager who manages the state management system 100 to provide an information service. The communication network is not limited to the Internet 4 and the like, and may be an independent communication network such as an intranet set up in a corporate network or a hospital.

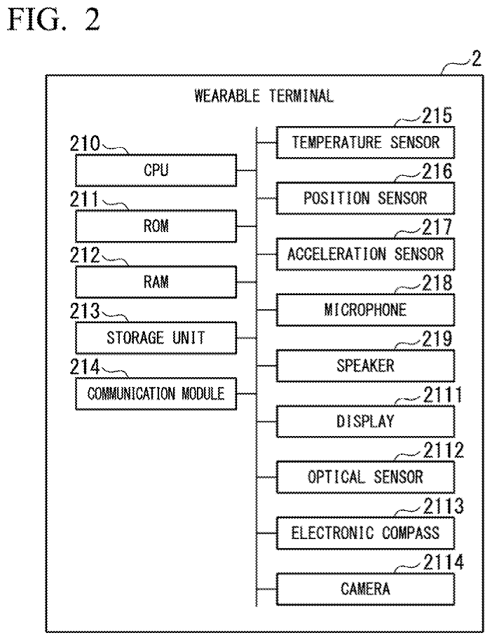

FIG. 2 is a diagram showing a hardware configuration of a wearable terminal.

More specifically, the wearable terminal 2 is an earphone 201, as an example. The earphone 201 may include a temperature sensor for measuring the body temperature of the user who is a patient, a position sensor for detecting the position of the user, an acceleration sensor, a microphone, a speaker, and the like. The earphone 201 can output from a speaker audio based on voice information transmitted from the state management device 1. The wearable terminal 2 may be a terminal device other than the earphone 201, for example, a head mounted device 202. Alternatively, the wearable terminal 2 may be a terminal device worn on clothing or the like, a watch-type terminal device worn on the user's arm, or the like.

In the present example embodiment, the wearable terminal 2 includes a CPU (Central Processing Unit) 210, a ROM (Read Only Memory) 211, a RAM (Random Access Memory) 212, a storage unit 213, a communication module 214, a temperature sensor 215, a position sensor 216, an acceleration sensor 217, a microphone 218, a speaker 219, and the like. The wearable terminal 2 may further include a display 2111, an optical sensor 2112, an electronic compass 2113, a camera 2114, and the like. The wearable terminal 2 can send sensing information acquired by the sensors such as the temperature sensor 215, the position sensor 216, the acceleration sensor 217, the microphone 218, the optical sensor 2112, the electronic compass 2113, and the camera 2114 to the state management device 1 using the communication module 214.

The sensing information of the temperature sensor 215 includes the body temperature of the user and the air temperature. The sensing information of the position sensor 216 is the user's position information. The sensing information of the acceleration sensor 217 is the acceleration of the user's movement. The sensing information of the microphone 218 is a sound emitted in the user's surrounding environment or the user's voice. The sensing information of the optical sensor 2112 is light generated in the user's surrounding environment. The sensing information of the electronic compass 2113 is the orientation of the user. The sensing information of the camera 2114 is an image (still image or moving image) taken of the user's surroundings. Of these types of sensing information, the user's body temperature and voice are examples of biometric information. Of the sensing information, the user's body temperature, voice, position information, acceleration, and user orientation are examples of state information. Of the sensing information, ambient sounds, light, and surrounding images of the user are examples of environmental information that affect the user who is the measurement subject. Further, the sensing information of the position sensor 216 may be position information acquired from a GPS (Global Positioning System), position information based on the base station with which the communication module 214 communicates, position information based on a wireless LAN access point with which the communication module 214 communicates, and the like.

The state management device 1 acquires biometric information and environmental information of the measurement subject from the wearable terminal 2 worn by the user who is the measurement subject, and based on the biometric information or environmental information and characteristics of the measurement subject, specifies stability promoting information that promotes the stability of the state of the user and outputs the stability promoting information to an output destination such as the wearable terminal 2.

Alternatively, the state management device 1 acquires the state information of the measurement subject from the wearable terminal 2 worn by the user who is the measurement subject, and on the basis of the state information and the characteristics of the measurement subject, provides a coping method that promotes the stability of the state of the user, and outputs the coping method to the output destination such as the wearable terminal 2.

The state management device 1 may directly transmit stability promoting information and state information to the wearable terminal 2. The state management device 1 may transmit the stability promoting information and the state information to the mobile terminal provided by the user, and the mobile terminal may transfer the stability promoting information and the state information to the wearable terminal 2.

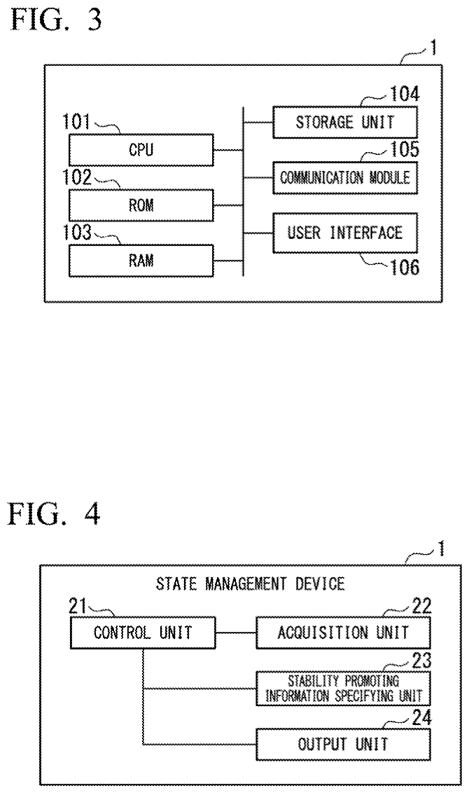

FIG. 3 is a diagram showing the hardware configuration of the state management device, the external device, and the mobile terminal.

Each of the state management device 1, the external device 3, and the mobile terminal 5 is a computer including hardware such as a CPU (Central Processing Unit) 101, a ROM (Read Only Memory) 102, a RAM (Random Access Memory) 103, a storage unit 104, a communication module 105 (appropriately called a communication unit), and a user interface 106. The state management device 1 and the external device 3 may internally or externally include a database as the storage unit 104.

FIG. 4 is a function block diagram of the state management device.

The state management device 1 executes a state management program stored in advance. As a result, the state management device 1 exhibits the functions of a control unit 21, an acquisition unit 22, a stability promoting information specifying unit 23, an output unit 24, and a treatment history database 25.

The control unit 21 controls other function units of the state management device 1.

The acquisition unit 22 acquires biometric information pertaining to a living body of the user to be measured, environmental information about the environment affecting the user (environment around the user), and state information pertaining to the state of the user from the wearable terminal 2 worn by the measurement subject.

The stability promoting information specifying unit 23 specifies the stability promoting information or coping method that promotes the stability of the state of the user on the basis of at least one of the biometric information of the user, the environment information around the user, and the user's state information, and the characteristics of the user.

The output unit 24 outputs the stability promoting information and information indicating a coping method to an output destination such as a wearable terminal 2.

The treatment history database 25 stores the treatment history information.

The external device 3 is, for example, a computer device that provides a weather service, and provides a service for outputting weather information at a position specified by the user. Alternatively, the external device 3 may be information that provides other services.

As an example, the state management device 1 according to the present example embodiment having the above-described configuration specifies music information related to a musical piece that serves as stability promoting information, and outputs the musical piece information (sound data of the musical piece) to the output destination. The state management device 1 also identifies drug information that serves as stability promoting information, and outputs the drug information to the output destination. The state management device 1 also identifies a coping method that serves as stability promoting information, and outputs the coping method to the output destination.

### First Example Embodiment

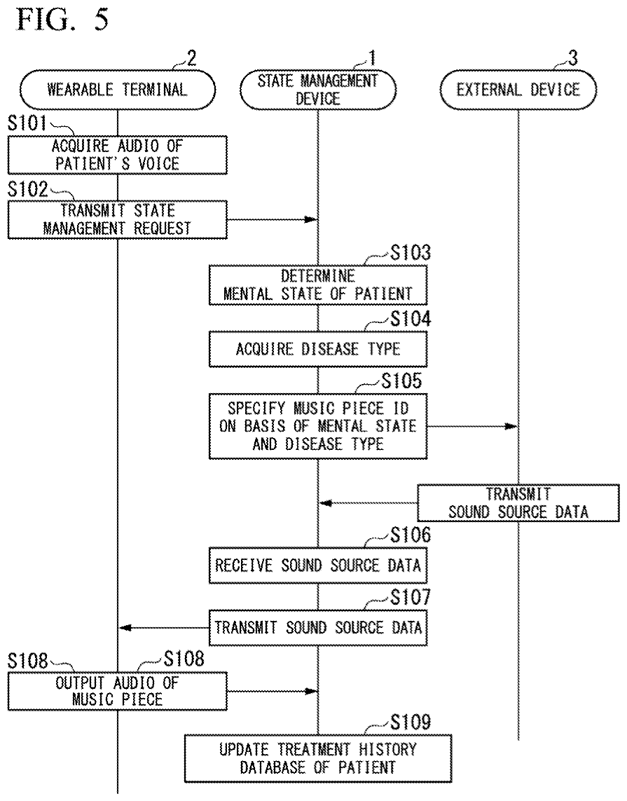

FIG. 5 is the first diagram showing the processing flow of the state management system according to the first example embodiment.

In the process in the state management system 100 of the first example embodiment, a process will be described for the case of the state management device 1 specifying stability promoting information that promotes stability of the state of the measurement subject on the basis of biometric information, environmental information, and characteristics of the measurement subject.

First, the CPU 210 of the wearable terminal 2 acquires the audio of the patient's voice from the microphone 218 as biometric information of the patient to be measured (Step S101). The CPU 210 generates a state management request including voice data acquired by converting the audio of a patient's voice into digital data and a user ID (user identifier) for identifying the patient. The CPU 210 outputs the state management request to the communication module 214. The communication module 214 then transmits the state management request to the state management device 1 (Step S102).

The state management device 1 receives the state management request. The acquisition unit 22 of the state management device 1 acquires the user ID and voice data from the state management request and outputs the user ID and voice data to the stability promoting information specifying unit 23. The stability promoting information specifying unit 23 analyzes the voice data and determines the mental state of the patient (Step S103). Specifically, the stability promoting information specifying unit 23 inputs the voice data into a mental state determination model generated by machine learning in the past, and as a result, calculates the mental state calculated based on the mental state determination model. For example, the stability promoting information specifying unit 23 may calculate the mental state based on other information held by the stability promoting information specifying unit 23. The other information includes weather information such as temperature and humidity, the season, and the time zone.

The stability promoting information specifying unit 23 acquires the disease type according to the user ID stored in advance in a storage unit such as a database (Step S104). When the stability promoting information specifying unit 23, as a result of examining effective treatment based on the mental state and disease type, past treatment response history, and the like, has determined that providing music is effective for stabilization of the patient's mental state, the stability promoting information specifying unit 23 specifies a music piece (music piece ID) that promotes stability of the patient's mental state from a plurality of music pieces (music piece information). More specifically, the stability promoting information specifying unit 23 inputs the patient's mental state and disease type into the music piece specifying model generated by machine learning in the past, and as a result specifies the music piece ID acquired using the music piece specifying model (Step S105). The stability promoting information specifying unit 23 acquires the sound source data of the specified music piece ID from the database. The external device 3 is a device capable of outputting sound source data of a music piece according to the music piece ID, and so the stability promoting information specifying unit 23 may transmit to the external device 3 the music piece ID corresponding to the specified music piece information, and as a result may receive from the external device 3 sound source data of the music piece (Step S106). The stability promoting information specifying unit 23 transmits the sound source data to the wearable terminal 2 (Step S107). Then, the wearable terminal 2 plays back the sound source data and outputs the audio of the music piece from the speaker 129 (Step S108).

According to the above processing, the state management device 1 transmits the sound source data of the music piece according to the patient's disease type and mental state to the wearable terminal 2, whereby the wearable terminal 2 can output the audio of that sound source data and promote the mental stability of the patient.

The wearable terminal 2 observes the user's state with respect to the provided music piece, and transmits the treatment history information including information on the effect and the user ID to the state management device 1. For example, the wearable terminal 2 acquires the user's state by a sensor, holds the information acquired by the sensor as the user's state, and transmits the information indicating the user's state by adding the information to the treatment history information. The state management device 1 receives the treatment history information pertaining to the user, and updates for retention information on the user recorded in the treatment history database with the newly received treatment history information (Step S109). This enables more appropriate treatment for the patient from the next time onward.

In the above process, voice data of the voice uttered by the patient as biometric information of the patient is included in the state management request transmitted by the wearable terminal 2 to the state management device 1, and the state management device 1 specifies music piece information which is stability promoting information of the patient on the basis of that state management request. However, other biometric information such as the patient's body temperature and heart rate may be used as the patient's biometric information to specify the stability promoting information, and state information such as the acceleration and position of the patient may be used for specifying stability promoting information instead of biometric information.

For example, the wearable terminal 2 transmits a state management request including at least one of body temperature and heart rate and a user ID to the state management device 1 in addition to or in place of the patient's voice. The stability promoting information specifying unit 23 of the state management device 1 inputs at least one of voice data indicating the patient's voice, body temperature, and heart rate into the mental state determination model, and as a result, specifies the patient's mental state on the basis of the mental state determination model. The stability promoting information specifying unit 23 inputs the specified mental state of the patient and the disease type of the patient into the music piece specifying model, and as a result, specifies the music piece ID acquired using the music piece specifying model.

Alternatively, in addition to or in place of the patient's voice, the wearable terminal 2 transmits a state management request including the patient's acceleration acquired from the accelerometer 217, the patient's position information acquired from the position sensor 216, at least one of a still image or moving image acquired from the camera 2114, and the user ID to the state management device 1. The stability promoting information specifying unit 23 of the state management device 1 inputs voice data indicating the patient's voice, acceleration, position information, and at least one of the still image and moving image into the mental state determination model, and as a result specifies the mental state of the patient on the basis of the mental state determination model. The stability promoting information specifying unit 23 inputs the specified mental state of the patient and disease type of the patient into the music piece specifying model, and as a result, specifies the music piece ID acquired using the music piece specifying model.

### Second Example Embodiment

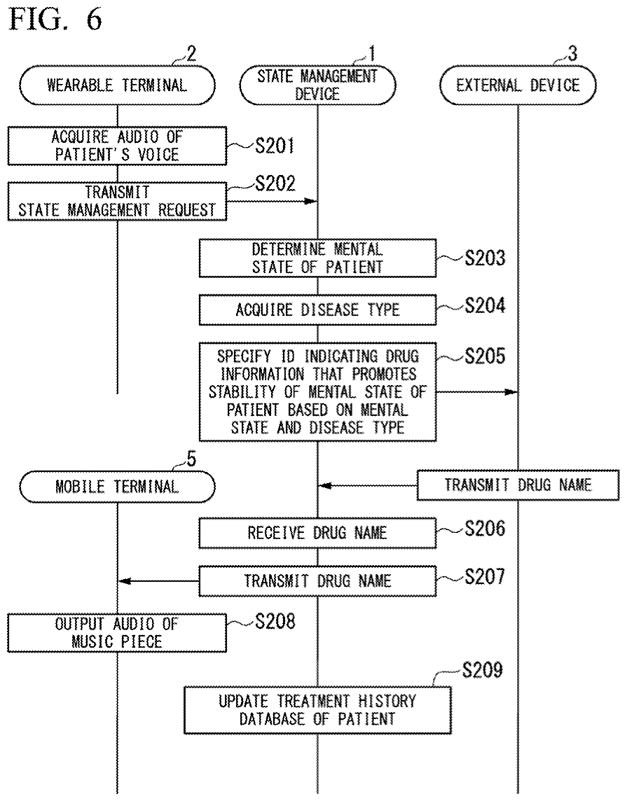

FIG. 6 is a diagram showing the processing flow of the state management system according to the second example embodiment.

In the processing of the first example embodiment, the stability promoting information specifying unit 23 specifies music piece information (music piece ID or sound source data) as stability promoting information that promotes the stability of the patient's state. However, in the processing of the second example embodiment, drug information and a coping method may be specified as stability promoting information that promotes stability of the patient's state.

The CPU 210 of the wearable terminal 2 acquires the audio of the patient's voice from the microphone 218 as biometric information of the patient to be measured (Step S201). The CPU 210 generates a state management request including voice data acquired by converting the audio of the patient's voice into digital data and a user ID for identifying the patient. The CPU 210 outputs the state management request to the communication module 214. The communication module 214 transmits the state management request to the state management device 1 (Step S202).

The state management device 1 receives the state management request. The acquisition unit 22 of the state management device 1 acquires the user ID and voice data from the state management request and outputs the user ID and voice data to the stability promoting information specifying unit 23. The stability promoting information specifying unit 23 analyzes the voice data and determines the mental state of the patient (Step S203). Specifically, the stability promoting information specifying unit 23 inputs the voice data into a mental state determination model generated by machine learning in the past, and as a result, calculates the mental state calculated based on the mental state determination model. For example, the stability promoting information specifying unit 23 may calculate the mental state based on other information held by the stability promoting information specifying unit 23. The other information includes weather information such as temperature and humidity, the season, and the time zone.

The stability promoting information specifying unit 23 acquires the disease type according to the user ID stored in advance in a storage unit such as a database (Step S204). The stability promoting information specifying unit 23 specifies drug information (ID indicating drug information) that promotes stability of the patient's mental state from a plurality of pieces of drug information on the basis of the mental state and the disease type (Step S205). More specifically, the stability promoting information specifying unit 23 inputs the patient's mental state and disease type into the drug specifying model generated by machine learning in the past, and as a result specifies the ID of the drug acquired using the drug specifying model. The stability promoting information specifying unit 23 acquires the drug name (name of the drug) corresponding to the ID of the specified drug from the database. The external device 3 is a device that outputs the drug name according to the ID of the drug, and so the stability promoting information specifying unit 23, as a result of transmitting to the external device 3 the specified drug ID, may as a result may receive the drug name corresponding to the drug ID from the external device 3 (Step S206). The stability promoting information specifying unit 23 transmits the drug name to the mobile terminal 5 carried by the patient's doctor, nurse, caregiver, or the like (Step S207). Then, the mobile terminal 5 displays the drug name on the display (Step S208).

The doctor, nurse, and caregiver observe the patient's progress after prescribing the drug, and input treatment history information including information on the effect of the drug into the mobile terminal 5. The mobile terminal 5 transmits the user ID of the patient and the treatment history information to the state management device 1. The state management device 1 receives the treatment history information pertaining to the patient (user), and updates for retention information on the patient (user) recorded in the treatment history database with the newly received treatment history information (Step S209). This enables more appropriate treatment for the patient from the next time onward.

According to the above processing, the state management device 1 transmits information on a drug according to the disease type and mental state of the patient to the mobile terminals 5 of the doctor, the nurse, and the caregiver, whereby it is possible to provide information on a drug that promotes mental stability of the patient.

### Third Example Embodiment

When the user to be measured is an infant, the stability promoting information specifying unit 23 of the state management device 1 may specify a coping method for the infant based on the voice data of the infant's voice. The coping method is, for example, a coping method such as giving milk or changing a diaper.

In this case, the stability promoting information specifying unit 23 may acquire a state management request including voice data and the user ID of the infant, input the voice data into an emotional state determination model, and calculate the emotional state. The stability promoting information specifying unit 23 inputs the emotional state and the type of user indicating the infant into the coping method specifying model, and as a result, specifies the coping method. In this case, the output unit 24 may transmit information indicating a coping method to the mobile terminal 5 carried by the parent of the infant. Upon receiving the information on the coping method, the mobile terminal 5 displays the information on the coping method in the display. The parent confirms the information on the coping method displayed in the mobile terminal 5 and provides care to the infant.

On the basis of the position information of the user, the state management device 1 may acquire information such as temperature, humidity, and atmospheric pressure from the external device 3, and further use the information to specify stability promoting information or a coping method of the user to be measured.

The above processing is described using an example when the measurement subject is a person. However, the measurement subject may be an animal such as livestock. For example, the wearable terminal 2 is attached to an animal. The wearable terminal 2 acquires information including at least one of biometric information of the animal (voice, body temperature, pulse, and the like), environmental information of the surroundings of the animal (temperature, sound, light, temperature, pressure, and the like), and state information such as position and acceleration of the animal (degree of movement), and transmits the information to the state management device 1. The stability promoting information specifying unit 23 of the state management device 1 specifies the stability promoting information and the coping method in the same manner as the above-mentioned processing. The state management device 1 transmits the stability promoting information and the coping method to the manager's mobile terminal or a device of a predetermined output destination. This makes it possible to manage the suitable condition of the animal. For example, the state management device 1 may specify temperature information for controlling the room temperature of the room of the animal as stability promoting information and transmit the information to a device such as an air conditioner or a fan. Further, the state management device 1 may specify music piece data of music that calms the movements of an animal as stability promoting information, and transmit the music piece data to a predetermined device such as a speaker or an audio device.

### Fourth Example Embodiment

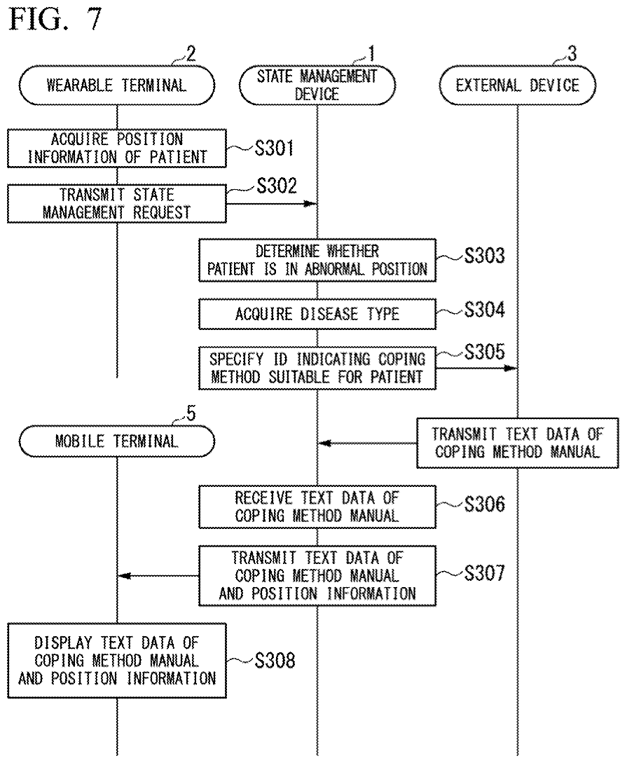

FIG. 7 is a diagram showing the processing flow of the state management system according to the fourth example embodiment.

In the state management system 100 of the fourth example embodiment, an example will be described for the case of the state management device 1, on the basis of the moving state of a patient, transmitting warning information promoting the stability of that moving state to a predetermined output destination device such as a mobile terminal 5.

As an example, some patients with dementia wander performs wandering behavior. The wearable terminal 2 is attached to such a patient. The CPU 210 of the wearable terminal 2 acquires the patient's position information from the position sensor 216 as the biometric information of the patient who is the measurement subject (Step S301). The position information may indicate an indoor position or an outdoor position. The CPU 210 generates a state management request including the position information of the patient and a user ID for identifying the patient. The CPU 210 outputs the state management request to the communication module 214. The communication module 214 transmits the state management request to the state management device 1 (Step S302).

The state management device 1 receives the state management request. The acquisition unit 22 of the state management device 1 acquires the user ID and the position information from the state management request and outputs the user ID and the position information to the stability promoting information specifying unit 23. The stability promoting information specifying unit 23 determines whether the patient is at an abnormal position on the basis of the position information (Step S303). For example, if the position of the patient is more than a predetermined distance from the hospital or home, a determination is made that the patient is in an abnormal position.

In addition to the position information, the wearable terminal 2 may transmit a state management request including the user's acceleration and body temperature to the state management device, and the stability promoting information specifying unit 23 of the state management device 1 may use information such as the position information, acceleration, and body temperature to determine whether or not the patient is in an abnormal position and in an abnormal physical condition. Specifically, the stability promoting information specifying unit 23 inputs information such as the position information, acceleration, and body temperature into the state stability determination model generated by machine learning in the past, and as a result, calculates the degree to which the patient is in an abnormal position and in an abnormal physical condition, calculated on the basis of the state stability determination model.

The stability promoting information specifying unit 23 acquires the disease type according to the user ID stored in advance in a storage unit such as a database (Step S304). The stability promoting information specifying unit 23 specifies a coping method suitable for the patient (ID indicating a coping method) from among a plurality of coping methods based on the degree of abnormality according to the distance from the predetermined position based on the abnormal position, the degree of abnormality calculated based on the state stability determination model, and the disease type (Step S305). More specifically, the stability promoting information specifying unit 23 inputs the degree of abnormality and the disease type (dementia and the degree of symptoms) of the patient into the coping method specifying model generated by machine learning in the past, and as a result, specifies the ID of the coping method acquired by using the coping method specifying model. The stability promoting information specifying unit 23 acquires text data of the coping method manual according to the ID of the specified coping method from the external device 3 (Step S306). The stability promoting information specifying unit 23 transmits the text data of the coping method manual and the position information to the mobile terminal 5 carried by the patient's doctor, nurse, caregiver, family, and the like (Step S307). Then, the mobile terminal 5 displays in the display the text data of the coping method manual and the position information of the patient (Step S308).

According to the above processing, the state management device 1 transmits the text data of the appropriate coping method manual according to the abnormal state of the patient to the mobile terminal 5 of the user assisting the patient, and thereby can promote the stability of the abnormal state of the patient.

The state management device 1 may transmit voice information pertaining to a voice that promotes control of the patient to the patient's wearable terminal 2 instead of the manual data of the coping method. For example, the stability promoting information specifying unit 23 acquires voice information such as words urging the patient to return to a predetermined place from a database according to the degree of abnormality of the patient and the type of disease. The stability promoting information specifying unit 23 transmits the voice information to the wearable terminal 2 worn by the patient. The wearable terminal 2 then outputs the voice information from the speaker.

The wearable terminal 2 transmits light information acquired by the optical sensor 2112 and a state management request including the orientation of the patient obtained by the electronic compass 211 to the state management device 1, and the state management device 1 may use those pieces of information to stabilize the degree of abnormality of the patient.

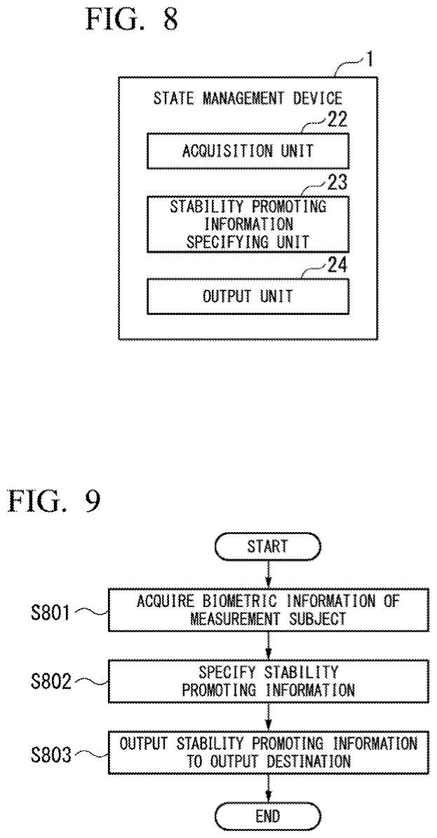

FIG. 8 is a diagram showing another configuration of the state management device.

FIG. 9 is a diagram showing a first processing flow by the state management device shown in FIG. 8.

As shown in FIG. 8, the state management device 1 includes at least an acquisition unit 22, a stability promoting information specifying unit 23, and an output unit 24.

The acquisition unit 22 acquires biometric information of the measurement subject from a wearable terminal worn by the measurement subject (Step S801).

The stability promoting information specifying unit 23 specifies stability promoting information that promotes stability of the state of the measurement subject on the basis of the biometric information and the characteristics of the measurement subject (Step S802).

The output unit 24 outputs the stability promoting information to the output destination (Step S803).

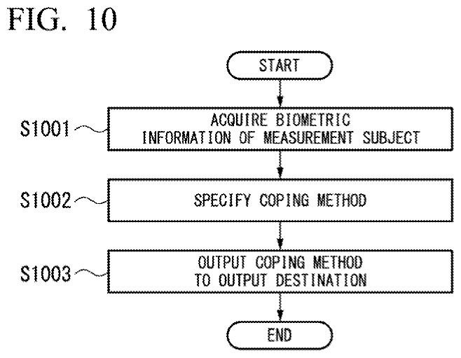

FIG. 10 is a diagram showing a second processing flow by the state management device shown in FIG. 8.

The acquisition unit 22 acquires the state information of the measurement subject from the wearable terminal 2 worn by the measurement subject (Step S1001).

The stability promoting information specifying unit 23 specifies a coping method for promoting the stability of the state of the measurement subject on the basis of the state information of the measurement subject and characteristics of the measurement subject (Step S1002). The coping method is one aspect of stability promoting information that promotes user stability.

The output unit 24 outputs the coping method to the output destination (Step S1003).

Each of the above-mentioned devices has a computer system inside. The process of each process described above is stored in a computer-readable recording medium in the form of a program, and the process is performed by the computer reading and executing this program. Here, the computer-readable recording medium means a magnetic disk, a magneto-optical disk, a CD-ROM, a DVD-ROM, a semiconductor memory, or the like. Further, this computer program may be distributed to a computer via a communication line, and the computer receiving the distribution may execute the program.

The aforementioned program may be used to implement some of the above-mentioned functions. In addition, the program may be a so-called differential file (differential program) that can implement the above-mentioned functions in combination with a program already recorded in the computer system.

### DESCRIPTION OF REFERENCE SYMBOLS

1: State management device
2: Wearable terminal
3: External device
4: Internet
5: Mobile terminal
201: Earphone
21: Control unit (control means)
22: Acquisition unit (acquisition means)
23: Stability promoting information specifying unit (stability promoting information specifying means)
24: Output unit (output means)

This application is based upon and claims the benefit of priority from Japanese patent application No. 2019-185118, filed Oct. 8, 2019, the disclosure of which is incorporated herein in its entirety by reference.

### INDUSTRIAL APPLICABILITY

The present invention may be applied to a state management device, a state management method, and a recording medium.

What is claimed is:

1. A state management device comprising:
at least one memory configured to store instructions; and
at least one processor configured to execute the instructions to:
acquire biometric information pertaining to a living body of a measurement subject from a wearable terminal worn by the measurement subject;
specify stability promoting information for promoting stability of a state of the measurement subject, based on the biometric information and a characteristic of the measurement subject; and
output the stability promoting information.

2. The state management device according to claim 1, wherein the at least one processor is configured to execute the instructions to:
acquire environmental information pertaining to an environment that affects the measurement subject; and
specify the stability promoting information based on the biometric information, the environmental information, and the characteristic of the measurement subject.

3. The state management device according to claim 1, wherein the at least one processor is configured to execute the instructions to:
specify music piece information pertaining to a music piece as the stability promoting information; and
output sound source data of the music piece.

4. The state management device according to claim 1, wherein the at least one processor is configured to execute the instructions to:
specify drug information pertaining to a drug as the stability promoting information; and
output the drug information.

5. The state management device according to claim 1, wherein the at least one processor is configured to execute the instructions to:
specify a coping method as the stability promoting information; and
output the coping method.

6. A state management device comprising:
at least one memory configured to store instructions; and
at least one processor configured to execute the instructions to:
acquire state information pertaining to a state of a measurement subject from a wearable terminal worn by the measurement subject;
specify a coping method for promoting stability of the state of the measurement subject, based on the state information and a characteristic of the measurement subject; and
output the coping method.

7. A state management method comprising:
acquiring biometric information pertaining to living body of a measurement subject from a wearable terminal worn by the measurement subject;
specifying stability promoting information for promoting stability of a state of the measurement subject, based on the biometric information and a characteristic of the measurement subject; and
outputting the stability promoting information.

8.-10. (canceled)
